(19) United States
(12) Patent Application Publication
(10) Pub. No.: US 2013/0085383 A1
(43) Pub. Date: Apr. 4, 2013

(54) SYSTEMS, METHODS AND COMPUTER READABLE STORAGE MEDIA STORING INSTRUCTIONS FOR IMAGE-GUIDED THERAPIES

(71) Applicant: Baowei Fei, Atlanta, GA (US)

(72) Inventor: Baowei Fei, Atlanta, GA (US)

(73) Assignee: EMORY UNIVERSITY, Atlanta, GA (US)

(21) Appl. No.: 13/613,440

(22) Filed: Sep. 13, 2012

Related U.S. Application Data
(60) Provisional application No. 61/542,902, filed on Oct. 4, 2011.

(51) Int. Cl. A61B 8/12; A61B 10/04; A61B 6/02; A61B 6/03; A61B 6/12; A61B 8/13

(52) U.S. Cl. 600/424

(57) ABSTRACT

Systems, methods, and computer-readable storage media relate to providing image guidance that can improve the accuracy of interventional procedures, such biopsy sampling, for example, by accurately providing location information to guide the biopsy needle image. The systems, methods, and computer-readable storage media include generating a 3D multi-modality image that includes a pre-procedure reference image and an ultrasound image; and displaying at least one view, one of the views including a display in-real-time of a position of the interventional device on the multi-modality image with respect to the target site during the procedure.

### CROSS REFERENCE TO RELATED APPLICATION

This application claims priority to U.S. Provisional Application No. 61/542,902 filed Oct. 4, 2011, hereby incorporated by reference in its entirety.

### ACKNOWLEDGEMENT

This invention was made with government support under Grant No. ROCA156775, awarded by the National Institutes of Health. The government has certain rights in the invention.

### BACKGROUND

Prostate cancer is the second leading cause of cancer death in men in the United States. It is estimated that there are 240,890 new cases and 33,720 deaths of prostate cancer the United States in 2011. See, e.g., Siegel et al., CA Cancer J Clin., 2011, 61 (4):212-36. Prostate specific antigen (PSA) measured via a blood test and digital rectal examination (DRE) are used to screen for prostate cancer, followed by a systematic transrectal ultrasound (TRUS) guided biopsy to confirm.

TRUS-guided biopsy is the clinical standard for definitive diagnosis of prostate cancer. Currently, two-dimensional (2D) ultrasound images are used to guide a biopsy needle to take tissue sample for pathological examination. However, this technique has a significant sampling error and is characterized by low sensitivity (39-52%). It has been reported that sextant biopsies can miss 30% of prostate cancers. This is a challenging problem for physicians as well as patients because a negative biopsy does not preclude the possibility of a missed cancer.

Additionally, 2D ultrasound imaging generally does provide accurate location information to guide the biopsy needle to lesion targets in three dimensions and differentiate carcinoma from benign prostate tissue.

### SUMMARY

Consequently, the physician must mentally estimate the 3D location of the biopsy needle based on limited 2D information, thus leading to suboptimal biopsy targeting. Thus, there is a need for an imaging processing techniques and systems that improve the accuracy of the biopsy sampling, for example, by accurately providing location information to guide the biopsy needle.

This disclosure generally relates to methods, systems, and computer readable storage media that for providing image guidance for a procedure at at least one target site using an interventional device.

In one embodiment, the system may include: an image guidance system configured to track the location of the interventional device with respect to the target site and configured to acquire an ultrasound image of an area surrounding the target site; an image processor configured to generate a 3D multi-modality image that includes a pre-procedure reference image and an ultrasound image; and a guidance module configured to display at least one view, one of the views including a display in-real-time of a position of the interventional device on the multi-modality image with respect to the target site during the procedure.

In some embodiments, the method may include acquiring an ultrasound image of an area surrounding the target site; generating a 3D multi-modality image that includes a pre-procedure reference image and the ultrasound image; and displaying at least one view, one of the views including a display in-real-time of a position of the interventional device on the multi-modality image with respect to the target site during the procedure.

In some embodiments, the computer readable medium may include instructions for: acquiring an ultrasound image of an area surrounding the target site; generating a 3D multi-modality image that includes a pre-procedure reference image and the ultrasound image; and displaying at least one view, one of the views including a display in-real-time of a position of the interventional device on the multi-modality image with respect to the target site during the procedure.

Additional advantages of the disclosure will be series forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 illustrates a system according to embodiments;

FIG. 2 illustrates an example of an image guidance system according to embodiments;

FIG. 3 illustrates a method according to embodiments for providing image guidance;

FIG. 4 shows an example of a fused image displayed for biopsy guidance;

FIG. 5 shows an example of deformable registration of CT and TRUS images; and

FIGS. 6(a)-(d) show examples of windows or views of the fused multi-modality images.

### DETAILED DESCRIPTION OF THE EMBODIMENTS

The following description, numerous specific details are series forth such as examples of specific components, devices, methods, etc., in order to provide an understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

This disclosure generally relates to methods, systems, and computer readable storage media that automatically reconstructs and displays the prostate image in 3D, in real-time, and that can be viewed in any dimension for enhanced tissue visualization. The methods, systems, and computer readable storage media are capable of detecting small tumors earlier, more precisely guiding therapy to cancer targets, and more accurately evaluating the therapeutic efficacy immediately after treatment. This will thereby provide physicians with both a powerful “eye and hand” to “see, treat, and evaluate” diseases, including cancers, and will thus be able to provide personalized therapy for each individual patient.

The disclosure is described with respect to an image-guided targeted biopsy of a prostrate and incorporating PET/CT images into TRUS images. However, it will be understood that the disclosure is not limited to image-guided targeted biopsy of a prostrate, biopsies and PET/CT. The system can also be used to for other organs, including, but not limited to, kidney, liver, and brain. The system can also be used to detect other cancers, including but not limited to, ovarian, liver, and breast cancers. The system can be used, for example, for other interventional procedures, such as image-guided therapies including, but not limited to, brachytherapy, radiofrequency thermal ablation, cryotherapy, photodynamic therapy, high intensity focused ultrasound (HIFU), and etc. Also, MRI/MRS (MR spectroscopy) can also be incorporated into the 3D ultrasound-guided system. By incorporating high-resolution, diffusion tensor MR imaging into the 3D ultrasound-guided procedures, the minimally invasive system may be able to spare the prostate nerve bundle and thus reduce the incidence of complications in those focal therapies.

As used in the disclosure, the term “real-time” refers to occurring at the same time, substantially at the same-time, and/or about the same time. For example, there may be a several second, up to about one minute, delay.

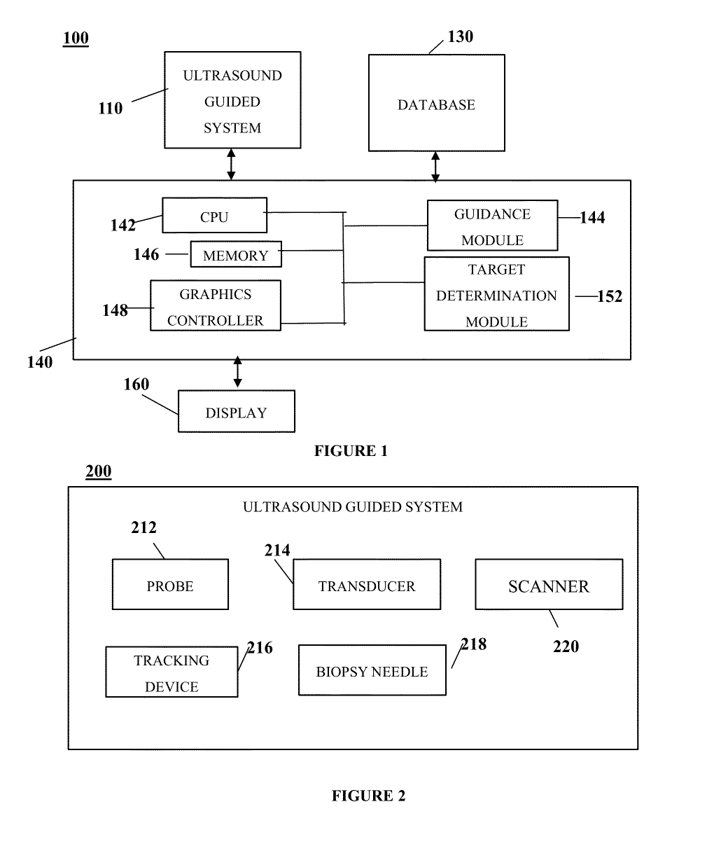

FIG. 1 shows an example of a system 100 according to embodiments. The system 100 may include an image guidance system 110. FIG. 2 shows an example of an image guidance system 200 configured for TRUS image guidance. It will be understood that the image guidance systems may be dependent on the organ to be treated and the type of treatment to be delivered.

The ultrasound guidance system 200 may include a probe 212 (an example of an interventional device), a transducer 214 that may be integrated with the probe 212, and a biopsy needle 218 that is configured to obtain prostate biopsies. The probe 212 may acquire plurality of individual images while being rotated through the area of interest (e.g., the prostate). The guidance system 210 may further include a tracking device 216 that is disposed on the probe 212, the transducer 214, and/or the biopsy needle 218, and that is configured to track placement of the probe 212, the transducer 214 and/or the biopsy needle 218. The tracking device 216 may be any sensor. The ultrasound guidance system 200 may include a scanner 220 configured to acquire ultrasound images, such as 2D images.

In some embodiments, the biopsy needle 218 may be a spring driven needle that is operated to obtain a core from desired area within the prostate. In certain embodiments, for example, for certain therapeutic procedures, guidance system 200 may not include a biopsy needle and the guidance system 200 may be operative to guide an introducer for a therapy device (e.g. guide arm) that allows for targeting tissue within the prostate. For example, the system 200 may provide guidance for an introducer (e.g., needle, trocar etc.) of a targeted focal therapy (TFT) device. TFT devices are generally configured to ablate cancer foci within the prostate using any one of a number of ablative modalities. These modalities may include, without limitation, cryotherapy, brachytherapy, targeted seed implantation, high-intensity focused ultrasound therapy (HIFU) and/or photodynamic therapy (PDT).

The system 100 may further include a database 130 configured to store raw and/or processed image data. The image data may be acquired from image modalities, that can include, but is not limited to, including ultrasound (US), magnetic resonance (MR), positron emission tomography (PET), computed tomography (CT), among others, or some combination thereof (i.e., PET/CT). The image data may be in Digital Imaging and Communications in Medicine (DICOM) format. The database 130 may also store ultrasound guided procedure images, for example, of a previous TRUS-guided biopsy.

The system 100 may further include a computer system 140 that is configured to run application software and computer programs that may control the image guidance system 110 components, provide a user interface, and/or cause the images to be displayed on a monitor 160. The computer system 100 may also perform the multimodal image fusion, guidance and tracking functionalities discussed herein.

In some embodiments, the computing system 140 may be a separate device. In other embodiments, the computing system 140 may be a part (e.g., stored on the memory) of other modules, for example, the image guidance system 110, and controlled by its respective CPUs.

The methods according to the present disclosure may be implemented as a routine that is stored in memory 146 and executed by CPU 142. As such, the computer system 140 may be a general purpose computer system that becomes a specific purpose computer system when executing the routine of the disclosure.

The system 140 may be a computing system, such as a workstation, computer, or the like. The system 140 may include one or more processors (e.g., central processing unit (CPU)) 142. The processor 142 may be one or more of any central processing units, including but not limited to a processor, or a microprocessor. The processor 142 may be coupled directly or indirectly to one or more computer-readable storage medium (e.g., physical memory) 146. The memory may include random access memory (RAM), read only memory (ROM), disk drive, tape drive, etc., or some combination thereof. The memory may also include a frame buffer for storing image data arrays. The memory 146 may be encoded or embed with computer-readable instructions, which, when executed by one or more processors 142 cause the system 140 to carry out various functions.

The system 140 may include a graphics controller 148. The graphics controller 148 may be one or more processors and/or microprocessors. The graphics controller 148 may be a part of and/or separate from the processor 142. The graphics controller 148 may be coupled directly or indirectly to one or more computer-readable storage medium (e.g., physical memory) 146. The memory may include random access memory (RAM), read only memory (ROM), disk drive, tape drive, etc., or some combination thereof. The memory may also include a frame buffer for storing image data arrays. The memory 146 may be encoded or embed with computer-readable instructions, which, when executed by graphics controller 148 cause the system 140 to carry out various functions. The graphics controller 148 may also be configured to process data for presentation on a monitor, such as display 160, in a human readable format.

The system 140 may include a guidance module 144. The guidance module 144 may be one or more processors and/or microprocessors. The guidance module 144 may be a part of and/or separate from the processor 142. The guidance module 144 may be configured to determine at least in part, accuracy of biopsies, position of the needle with respect to the target, among others, or some combination thereof. The guidance module may be configured to process the images and data to cause a display of one or more windows (discussed in below).

The system 140 may include a target determination module 152. The target determination module 152 may be a part of and/or separate from the processor 142 and/or guidance module 144. The target determination module 152 may be configured to determine at least one target site for a biopsy based on at least in part, the tumor information provided on the PET image, previous biopsy locations, or some combination thereof. The target determination module 152 may further include a biopsy accuracy determination module configured to determine accuracy of the biopsy with respect to the planned target site.

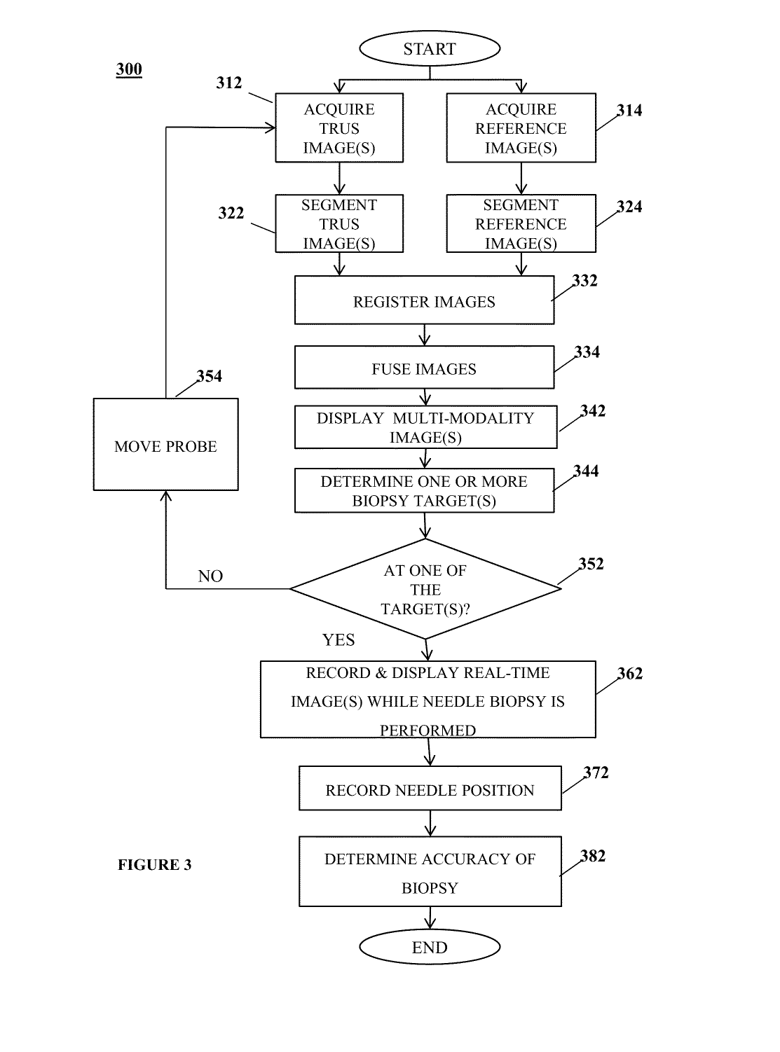

FIG. 3 shows a method 300 of generating 3D ultrasound guidance according to embodiments. The methods of the disclosure are not limited to the steps described herein. The steps may be individually modified or omitted, as well as additional steps may be added.

Unless stated otherwise as apparent from the following discussion, it will be appreciated that terms such as “identifying,” “fusing,” “recording,” “measuring,” “receiving,” “integrating,” “filtering,” “combining,” “reconstructing,” “segmenting,” “generating,” “registering,” “determining,” “obtaining,” “processing,” “computing,” “selecting,” “estimating,” “acquiring,” “detecting,” “tracking,” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure.

The method 300 may include a step 312 of acquiring an ultrasound image (TRUS) of a prostate of a patient. The step 312 may include acquiring a first ultrasound image of a prostate of the patient. The first ultrasound image can be before the procedure (e.g., biopsy) is performed. The ultrasound image may be 2D images that are reconstructed into a 3D image. The images may be acquired by rotating the transducer about its long axis. In some embodiments, the 3D scan may be composed of 200 images in 1-deg increments of the prostate.

Additionally, before the first 3D image of the prostate, the tracking arm can be locked into place to prevent the TRUS probe from changing its pitch, yaw, and depth of penetration while the probe is being rotated. As the physician rotates the transducer manually about its long axis to acquire a 3D scan composed of, for example, about 200 images in 1-deg increments of the prostate, the 2D TRUS images from the ultrasound machine can be digitized using a frame grabber and are reconstructed into a 3D image by a graphics controller 148. In some embodiments, the last group of images (e.g., 20 images) in the overlap region from, for example, 180 deg to 200 deg and 0 deg to 20 deg, of transducer rotation can be merged by averaging the duplicate images in order to remove any slight discontinuities that may arise from image lag or a small amount of patient motion. The graphics controller 148 can be configured to provide information to the physician regarding the proper rotation speed.

The system 100 can provide a visual queue to indicate whether the rotation was too rapid and the 2D TRUS images were not properly acquired. Once the 3D TRUS image has been acquired, the image can be displayed in a cube view.

Next, the TRUS image may be segmented (step 322). The TRUS image may be segmented according to any segmentation methods. In some embodiments, the TRUS image may be segmented by the method, for example, disclosed by International Application No. PCT/US2012/024844, which is hereby incorporated by reference in its entirety.

The method 300 may include a step 314 of acquiring one or more reference images, for example, from a storage device 130. In some embodiments, the reference images may include pre-biopsy images acquired by imaging modalities other than ultrasound. In some embodiments, the imaging modalities may include but is not limited to PET/CT and MR. In certain embodiments, the reference images may alternatively and/or additionally include previous biopsy images.

In some embodiment, the method 300 may include a step 324 of segmenting the reference images. The segmenting may be according to any segmentation techniques.

Next, the reference and TRUS images may be registered (step 332). The reference and TRUS images may be registered by any registration techniques. For example, if the references images are PET/CT and/or MR, the registration may be based on the technique disclosed in International Application No. PCT/US2012/024821, which is hereby incorporated in its entirety.

In some embodiments, if the reference images are PET/CT, the step of registering the TRUS and reference images may first include transforming an individual's pre-biopsy CT to the TRUS images, and then applying the same transformation to the PET volume.

In some embodiments, this transformation may include into three consecutive motions: (i) TCT-Atlas, the transformation from a new CT to the CT atlas, (ii) Tatlas-atlas, the transformation from the CT atlas to the TRUS atlas, and (iii) TAtlas-TRUS, the transformation from the TRUS atlas to the new TRUS, as shown in FIG. 5. After the prostate is segmented on CT and 3D TRUS images, the prostate surface registration can become to solve B-spline motion from CT→CT atlas→TRUS atlas→TRUS.

Then, the same transformation may be applied to the PET volume that has been aligned with CT. The PET image can in turn be registered and fused with TRUS to provide the location of suspicious cancer sites with a high metabolic activity seen on PET images. These sites can be automatically determined from PET data and thus offer biopsy targets. PET would provide complementary information for the ultrasound imaging guidance. Each registration can be visually evaluated and confirmed. Segmented surfaces from CT can be overlapped with the surface from 3D TRUS. The overlapping ratio can provide a quantitative measurement of the surface registration. Meanwhile, the fusion of CT with 3D TRUS can visually illustrate the registration quality.

Next, the reference and 3D TRUS images may be fused to generate at least one multi-modality image (step 334). During the biopsy procedure, 2D ultrasound images can be acquired and fused with a PET slice in real-time. Because the probe may be secured on the mechanical tracking system and only rotation and sliding along its long axis can be allowed, the displacement between the real-time 2D TRUS image and the 3D TRUS volume will be quite small. Also, image and volume can be easily registered, using, for example, a slice-to-volume registration method. See, for example, Baowei Fei, Jeffrey L. Duerk, Nakiel T. Boll, Jonathan S. Lewin, David L. Wilson: Slice to Volume Registration and its Potential Application to Interventional MRI Guided Radiofrequency Thermal Ablation of Prostate Cancer. IEEE Trans. Med. Imaging 22 (4): 515-525 (2003), which is hereby incorporated by reference.

According to the methods of the embodiment, the prostate boundary in the 2D TRUS can be segmented in about real-time and can be compared with the boundary on the slice from the aligned 3D TRUS. In this way, it can be determined whether the prostate moves during the procedure and whether another 3D TRUS scan should be performed during the same examination. This decision can be made by either visual inspection of the two boundaries or by automatic comparison of the boundary distances using a given criterion. As the 3D TRUS has been registered with the PET/CT volume, the PET image can be, in turn, be fused with the real-time TRUS images. The tumor information on the PET image can be the target of the needle biopsy.

The images may be displayed on a display, for example, display 160. The displayed images may include an integrated image of the 3D rendering of the prostate, suspicious tumors delineated from PET, the bladder, and the rectum. The integrated image may be displayed on a main window provided on the display 160 to provide an interactive scene to guide the biopsy. Also, the needle position may also be displayed on the images.

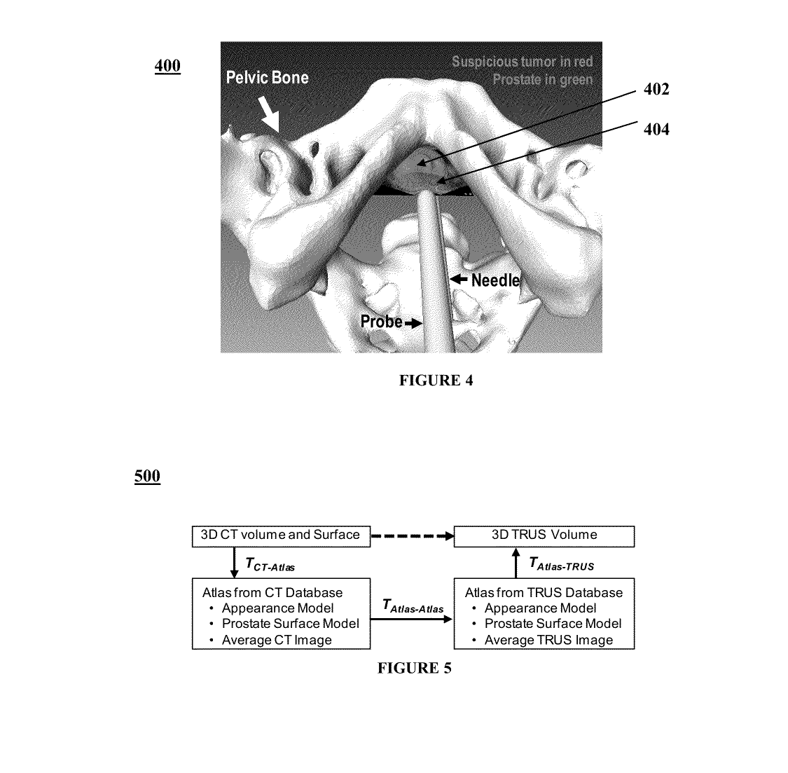

During the procedure of manipulating the probe towards a target location, real-time TRUS, corresponding PET, and fused images can be displayed for biopsy guidance, for example, as shown in FIG. 4. A main window showing the whole scene can also intuitively illustrate the probe motion and the distance to the correct plane containing the biopsy target. This information can also be provided on the display. The displayed images provided on the main window can be configured to be freely zoomed in/out, rotated and translated in the 3D space so a physician can easily determine how to operate the probe in order to reach the target position. The projection of the scene along the probe direction can also show the current needle position and the trajectory.

Next, one or more biopsy target sites may be determined (step 344). The biopsy targets may be automatically determined based on at least in part, the tumor information provided on the PET image, previous biopsy locations, among other information, or some combination thereof.

Once the 3D image scanning and biopsy plan are complete, the system can display a 3D needle guidance interface to facilitate the systematic targeting of each biopsy site location. Throughout the biopsy procedure, the 3D location and orientation of the TRUS transducer can be tracked and displayed in real-time on at least the multi-modality image. The displayed images may include a biopsy interface.

In some embodiments, the biopsy interface may include a plurality of windows or views. In some embodiments, the windows or views may include at least one 2D TRUS window or view that includes real-time 2D TRUS video stream, at least one 3D TRUS window or view that includes at least one real-time 3D multi-modality image, and at least one 3D TRUS targeting window or view. The 2D TRUS window may display the real-time 2D TRUS image streamed from the ultrasound machine. The 3D TRUS window may include the 3D TRUS image sliced in real-time to correspond to the orientation and position of the TRUS probe. This correspondence can allow the physician to compare the static 3D image with the real-time 2D image. The at least 3D TRUS targeting view may include one or more views, each showing the coronal and/or perspective views of the 3D prostate model, the real-time position of the 2D TRUS image plane, and the expected path of the biopsy needle as defined by the biopsy guide.

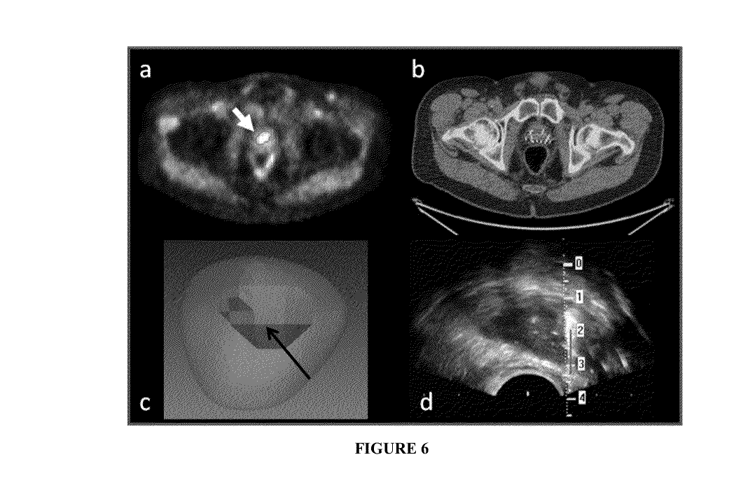

FIGS. 6(a)-(d) show examples of the images used to generate the multi-modality image and that may be provided in the windows or views. FIG. 6(a) shows an FACBC PET image (a PET image acquired by using a FACBC PET tracer). The image also shows the suspicious lesion (illustrated by an arrow). FIG. 6(b) shows the combined PET/CT images in which the prostate was segmented. FIG. 6(c) shows the 3D fused multi-modality image of the prostate and the lesion for biopsy planning (illustrated by an arrow). FIG. 6(d) shows a real-time ultrasound image that captured the needle path during the biopsy gun firing.

The method may further include a determination of whether the needle is located at one of the biopsy target sites (step 352). The determination may include determining distance between the needle and the planned target site. If it is determined that the probe is not located at the target site and the probe is moved (step 354), the display (step 342) may be updated by processing the new TRUS image, for example, according to steps 312, 322, 332 and 334 based on the new location of the probe. In some embodiments, the fused multi-modality image may be updated, for example, by segmenting and registering the new TRUS image to the pre-biopsy image. It will be understood that the display (step 342) may be updated anytime the probe is moved, and that the probe may be moved anytime during the procedure (e.g., it is not limited to step 354).

Next, while the needle biopsy or other procedure is performed, the TRUS images may be displayed and recorded in real-time (step 362). After the biopsy or other procedure has been completed, the needle position may be recorded (step 372) and the accuracy of the biopsy or procedure may be determined (step 382). The images may be recorded in a computer-readable storage medium, for example, memory 146 and/or the database 130.

In some embodiments, previous biopsy locations may be displayed. In some embodiments, the biopsy needle may be rendered as cylindrical shapes to illustrate previous biopsy locations. However, it will be understood that any shape or symbol may be used to illustrate previous biopsy locations.

In some embodiments, the accuracy of the biopsy may be determined based on location of the needle with respect to the planned target site(s). The needle insertion can be monitored by real-time TRUS. The final biopsy location can be recorded by recognizing the needle in the TRUS image. This may be implemented by subtracting consecutive TRUS images as only the moving needle has a changing signal. Both the actual biopsy location(s) and the planned site(s) may be displayed in the main window. The accuracy of the biopsy may be determined based on the distance between the actual biopsy location and the planned site.

In some embodiments, one or more steps, for example, steps 352 through 382, may be repeated for each planned biopsy target site. In other embodiments, step 382 may be performed after all the steps 352 through 372 have been performed for all of the targets sites. It will also be understood that step 344 of determining one or more biopsy target sites may not need to be repeated after the initial determination of the one or more biopsy target sites.

According to embodiments, the system 100 can enable real-time response to user interaction. The system can offer a unique but friendly tool for performing a reliable and accurate prostate biopsy with the aid of the fused metabolic and anatomic information obtained from another modality, such as PET/CT.

The computer system 140 may also include an operating system and micro instruction code. The various processes and functions described herein may either be part of the micro instruction code or part of the application program or routine (or combination thereof) that is executed via the operating system. In addition, various other peripheral devices may be connected to the computer platform such as an additional data storage device, a printing device, and I/O devices.

It is also to be understood that the system may omit any of the modules illustrated and/or may include additional modules not shown. It is also be understood that more than one module may be part of the system although one of each module is illustrated in the system. It is further to be understood that each of the plurality of modules may be different or may be the same. It is also to be understood that the modules may omit any of the components illustrated and/or may include additional component(s) not shown.

In some embodiments, the modules provided within the system may be time synchronized. In further embodiments, the system may be time synchronized with other systems, such as those systems that may be on the medical facility network.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that, because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

While the disclosure has been described in detail with reference to exemplary embodiments, those skilled in the art will appreciate that various modifications and substitutions may be made thereto without departing from the spirit and scope of the disclosure as series forth in the appended claims. For example, elements and/or features of different exemplary embodiments may be combined with each other and/or substituted for each other within the scope of this disclosure and appended claims.

What is claimed is:

1. A system for providing image guidance for a procedure at at least one target site using an interventional device comprising:
an image guidance system configured to track the location of the interventional device with respect to the target site and configured to acquire an ultrasound image of an area surrounding the target site;
an image processor configured to generate a 3D multi-modality image that includes a pre-procedure reference image and an ultrasound image; and
a guidance module configured to display at least one view, one of the views including a display in-real-time of a position of the interventional device on the multi-modality image with respect to the target site during the procedure.

2. The system according to claim 1, wherein the pre-procedure reference image is a PET/CT image acquired before the procedure.

3. The system according to claim 2, wherein the guidance module is configured to register the PET image to the ultrasound image based on the registration of the CT image to the ultrasound image.

4. The system according to claim 1, wherein:
a plurality of views are displayed;
the plurality of views includes at least one 2D TRUS view that includes real-time 2D TRUS video stream, at least one 3D TRUS view that includes at least one real-time 3D multi-modality image, and at least one 3D TRUS targeting view;
the 2D TRUS view may be streamed from an ultrasound machine;
the 3D TRUS view may include the 3D TRUS image sliced in real-time to correspond to the orientation and position of the interventional device; and
the at least one 3D targeting view may include at least one of coronal and perspective views of a 3D model of the area, real-time position of 2D TRUS image plane, and expected path of the interventional device.

5. The system according to claim 1, wherein the ultrasound image is acquired by a systematic transrectal ultrasound (TRUS) system and the procedure is a biopsy of a prostate.

6. The system according to claim 1, further comprising:
a target determination module configured to determine at least target site for a biopsy based on at least in part, the tumor information provided on the PET image, previous biopsy locations, or some combination thereof.

7. The system according to claim 6, wherein the guidance module is configured to display motion of the interventional device and a distance to the target site.

8. The system according to claim 6, further comprising:
a biopsy accuracy determination module configured to determine accuracy of the biopsy with respect to the planned target site.

9. The system according to claim 8, wherein the accuracy is determined based on location of the needle with respect to the planned target site.

10. A method for providing image guidance for a procedure at at least one target site using an interventional device, comprising:
acquiring an ultrasound image of an area surrounding the target site;
generating a 3D multi-modality image that includes a pre-procedure reference image and the ultrasound image; and
displaying at least one view, one of the views including a display in-real-time of a position of the interventional device on the multi-modality image with respect to the target site during the procedure.

11. The method according to claim 10, wherein the pre-procedure reference image is a PET/CT image acquired before the procedure.

12. The method according to claim 10, wherein the plurality of views includes the plurality of views includes at least one 2D TRUS view that includes real-time 2D TRUS video stream, at least one 3D TRUS view that includes at least one real-time 3D multi-modality image, and at least one 3D TRUS targeting view.

13. The method according to claim 10, wherein the ultrasound image is acquired by a systematic transrectal ultrasound (TRUS) system and the procedure is a biopsy of the prostate.

14. The method according to claim 13, further comprising:
determining at least one target site for the procedure determined based on at least in part, the tumor information provided on the PET image, previous biopsy locations, or some combination thereof.

15. The method according to claim 1, wherein the displaying includes displaying motion of the interventional device and a distance to the target site.

16. A computer-readable storage medium storing instructions for providing image guidance for a procedure at at least one target site using an interventional device, the instructions comprising:
acquiring an ultrasound image of an area surrounding the target site;
generating a 3D multi-modality image that includes a pre-procedure reference image and the ultrasound image; and
displaying at least one view, one of the views including a display in-real-time of a position of the interventional device on the multi-modality image with respect to the target site during the procedure.

17. The medium according to claim 16, wherein the pre-procedure reference image is a PET/CT image acquired before the procedure.

18. The medium according to claim 16, wherein the plurality of views includes the plurality of views includes at least one 2D TRUS view that includes real-time 2D TRUS video stream, at least one 3D TRUS view that includes at least one real-time 3D multi-modality image, and at least one 3D TRUS targeting view.

19. The medium according to claim 16, wherein the ultrasound image is acquired by a systematic transrectal ultrasound (TRUS) system and the procedure is a biopsy of the prostate.

20. The medium according to claim 16, further comprising instructions for:
determining at least one target site for the procedure determined based on at least in part, the tumor information provided on the PET image, previous biopsy locations, or some combination thereof.
